(19) United States
(12) Patent Application Publication
(10) Pub. No.: US 2021/0396673 A1
(43) Pub. Date: Dec. 23, 2021

(54) A METHOD FOR DETERMINING HYDROLYSIS DEGREE AND CHARGE DENSITY OF POLYELECTROLYTES AND PHOSPHONATES

(71) Applicant: Kemira Oyj, Helsinki (FI)

(72) Inventors: Salla PUUPPONEN, Espoo (FI); Susanna TOIVONEN, Espoo (FI); Sari KRAPU, Espoo (FI)

(21) Appl. No.: 17/281,601

(22) PCT Filed: Sep. 27, 2019
(86) PCT No.: PCT/FI2019/050693

§ 371 Date: Mar. 31, 2021

(30) Foreign Application Priority Data
Oct. 1, 2018 (FI) 20185815

(51) Int. Cl. G01N 21/64; G01N 21/77; G01N 33/44; G01N 1/38

(52) U.S. Cl. CPC G01N 21/6408; G01N 21/77; G01N 2001/386; G01N 1/38; G01N 2021/6497; G01N 33/442

(57) ABSTRACT

The present invention relates to a method for determining hydrolysis degree and charge density of polyelectrolyte or phosphonate in a sample. In the method the sample is mixed with a reagent comprising a lanthanide(lll) ion. The mixture is excited at an excitation wavelength and a signal deriving from the lanthanide(lll) ion is detected by using time-resolved fluorescence measurement, followed by determining the hydrolysis degree and the charge density of the polyelectrolyte or phosphonate by using the detected sample signal.

### FIELD OF THE INVENTION

The present invention relates to a method for determining hydrolysis degree or charge density of polyelectrolytes and phosphonates. The invention further relates to use of the method for determining hydrolysis degree or charge density of polyelectrolyte or phosphonate in samples originating from various processes.

### BACK GROUND

Polyelectrolytes are used in various industries, such as in water treatment, paper and oil industry. Many critical properties of polyelectrolytes, such as conformation, geometry, conductivity, viscosity and precipitation tendency are based on the charge density of the polyelectrolytes.

As many important properties of the polyelectrolytes depend strongly on their charging, it is important to be able to measure polymer charge in an easy and quick manner. For instance, in enhanced oil recovery (EOR), polyacrylamide polymers are commonly used for increasing the viscosity of the injected water in the oil reservoir to push the oil towards the production well. The main properties (viscosity, precipitation tendency, conformation) of the polyelectrolyte, such as polyacrylamide, used depend strongly on the hydrolysis degree of polyacrylamide.

It is also important to control the hydrolysis degree of the injected polymer, ranging often from 20 mol-% to 40 mol-%. The hydrolysis degree measurement of produced polymers is often also important, as the polymer may hydrolyze further in the wells in high temperature resulting in changes in the polymer properties.

In enhanced oil recovery applications polymers, such as polyamides or phosphonates, are often susceptible to chemical, thermal, and mechanical degradation. Chemical degradation occurs when the labile moieties, such as amide or phosphonate groups, hydrolyze at elevated temperature or acidic/basic environment.

Viscosity of the hydrolyzed polymer is often significantly lower than viscosity of the non-hydrolyzed polymer. The hydrolysis degree has also effect on the charge density of the polymer and therefore on precipitation tendency.

Decrease of the viscosity of the polymer will, thus, also decrease viscosity of the aqueous solution which can generate problems in enhanced oil recovery processes. Therefore, it is essential to monitor the hydrolysis degree of polymer in the process.

There have been developed methods for determining hydrolysis degree of polyelectrolytes. An example of such method is potentiometric titration of polymer with HCl and NaOH. From the titration curves and control samples it is possible to determine level of hydrolysis.

However, there is still need for new, simpler and more efficient methods for determining hydrolysis degree and charge density of polyelectrolytes and phosphonates in a sample.

### SUMMARY OF INVENTION

An object of the present invention is to provide a method for determining hydrolysis degree and charge density of polyelectrolytes and phosphonates.

Another object of the present invention is to provide a simple and efficient method for determining hydrolysis and charge density of polyelectrolytes and phosphonates in a sample.

The inventors surprisingly found that luminescence signal intensity of lanthanide(III) ions depends on charge density and hydrolysis degree of the polyelectrolyte and phosphonate acting as the chelating agent. This observation can be utilized for measurement of charge density or hydrolysis degree of several types of polyelectrolytes, such as polyamides, polycarboxylates or polyamines and phosphonates.

The luminescence signal intensity of the lanthanide (III) ion depends on hydrolysis degree of the polyelectrolyte or the phosphonate. Therefore, in one embodiment of the present invention, hydrolysis degree of the polyelectrolyte and the phosphonate is measured with time-resolved luminescence method, such as time-resolved fluorescence (TRF).

The luminescence signal intensity of the lanthanide (III) ion depends on charge density of the polyelectrolyte or the phosphonate. Therefore, in other embodiment of the present invention, charge density of the polyelectrolyte or the phosphonate is measured with time-resolved luminescence method, such as TRF.

The luminescence signal from unknown sample comprising polyelectrolyte or phosphonate can be compared with luminescence signals of known samples comprising polyelectrolytes or the phosphonates having different hydrolysis degrees or charge densities (calibration curves) in order to determine the hydrolysis degree or the charge density of the polyelectrolyte or the phosphonate in the sample.

The present invention provides a simple and effective method to determine hydrolysis degree or charge density of polyelectrolyte or phosphonate in a sample. The method comprises
optionally diluting and/or purifying the sample,
admixing the sample with a reagent comprising a lanthanide(III) ion,
allowing the polyelectrolyte or phosphonate in the sample to interact with the reagent comprising the lanthanide(III) ion,
exciting the sample at an excitation wavelength and detecting a sample signal deriving from the lanthanide(III) ion at a signal wavelength by using time-resolved fluorescence measurement, and
determining the hydrolysis degree or the charge density of the polyelectrolyte or phosphonate in the sample by using the detected sample signal

The method may be utilized to determine hydrolysis degree or charge density of polyelectrolyte or phosphonate in a sample originating, for example, from water treatment and paper making processes as well as from pharmaceutical industry, well drilling, mineral processing and enhanced oil recovery.

### BRIEF DESCRIPTION OF THE DRAWINGS

FIG. 1 illustrates TRF response of polyacrylamides with different hydrolysis degree.

FIG. 2 illustrates TRF signal as a function of hydrolysis degree of polyacrylamide.

FIG. 3 illustrates TRF signals of polyacrylic acid and sodium polyacrylate for determining charge densities.

### DETAILED DESCRIPTION

The present invention relates to a method for determining hydrolysis degree or charge density of polyelectrolyte or phosphonate in a sample. More particularly the present invention relates to a method for determining hydrolysis degree or charge density of polyelectrolyte or phosphonate in a sample comprising polyelectrolyte or phosphonate, the method comprising
optionally diluting and/or purifying the sample,
admixing the sample with a reagent comprising a lanthanide(III) ion,
allowing the polyelectrolyte or phosphonate in the sample to interact with the reagent comprising the lanthanide(III) ion,
exciting the sample at an excitation wavelength and detecting a sample signal deriving from the lanthanide(III) ion at a signal wavelength by using time-resolved fluorescence measurement, and
determining the hydrolysis degree or the charge density of the polyelectrolyte or phosphonate in the sample by using the detected sample signal.

The analyte, polyelectrolyte or phosphonate, in the sample bears one or more groups that can hydrolyze and/or that the sample bears one or more groups that are capable of dissociating in aqueous solution to form either anion or cation groups. The analyte can be zwitterionic, i.e. contain both cationic and anionic groups. The charging of the group can depend on the environment pH (acidic or basic groups, such as carboxylic acids and amino groups). Therefore, the groups capable for dissociation can be neutral in certain pH (e.g. carboxylic acid group in acidic and amino groups in basic environment). The polyelectrolyte can be basic or acidic.

In one embodiment the polyelectrolyte contains one or more groups selected from, carboxylic acid/carboxylate, amide, phosphonate, amine, or any combination thereof.

In a preferred method the polyelectrolyte contain aromatic groups. The aromatic group(s) amplify the signal of lanthanide(III) ion.

In one embodiment the polyelectrolyte has molecular weight of at least 1000 g/mol.

In another embodiment the phosphonate has molecular weight of at least 100 g/mol.

The lanthanide(III) ion is selected from europium, terbium, samarium or dysprosium ions, preferably europium or terbium ions.

In a preferred embodiment the lanthanide(III) ion is a lanthanide(III) salt. The lanthanide(III) salt is selected from halogenides and oxyanions, such as nitrates, sulfates or carbonates, preferably from hydrated halogenides or nitrates, more preferably hydrated chloride.

In one embodiment the method of the present invention is utilized for determining charge density of polyelectrolytes or phosphonates. In another embodiment the method of the present invention is utilized for determining of hydrolysis degree of polyelectrolytes such as amide containing polyelectrolytes or phosphonates.

In one embodiment hydrolysis degree or charge density of polyelectrolyte is determined.

In other embodiment hydrolysis degree or charge density of phosphonate is determined.

The sample may be optionally diluted or purified prior mixing the sample with the reagent comprising a lanthanide(III) ion.

The sample is optionally purified by using a purification method selected from centrifugation, size exclusion chromatography, cleaning with solid-phase extraction (SPE) cartridges, dialysis techniques, extraction methods for removing hydrocarbons, filtration, microfiltration, ultrafiltration, nanofiltration, membrane centrifugation and any combinations thereof. It should be understood that the purification treatment step means preferably removal or dilution of molecules that may disturb the examination of charged molecules of interest, not isolation of the polyelectrolyte e.g. by chromatography.

The polyelectrolyte or phosphonate is optionally diluted to suitable aqueous solution e.g. deionized water or brine containing monovalent and/or divalent ions. Preferably, the dissolution brine does not contain any trivalent ions. If the polymer solution contains some interfering compounds, suitable pretreatment procedures may be applied prior to the dilution steps. Preferably the sample is an aqueous solution.

In one embodiment concentration of the lanthanide(III) ion in the measurement mixture comprising the sample and the lanthanide (III) ion is in range of 0.1-100 μM, preferably 0.1-20 μM, and more preferably 1-20 μM.

In other embodiment concentration of the polyelectrolyte or phosphonate in the measurement mixture comprising the sample and the lanthanide (III) ion is in range of 0.01-100 ppm, preferably 0.5-50 ppm, and more preferably 0.5-20 ppm.

In case the concentration of the polyelectrolyte in the sample is higher, the sample can be diluted

By term “measurement mixture” is meant the admixture in the measurement.

In one embodiment a signal modifier is added to the sample before the excitation of the sample. The signal modifier comprises a metal ion selected from a group comprising copper, nickel, chromium, iron, gold, silver, cobalt, and any of their mixtures.

In one embodiment a pH value of the sample is adjusted prior the mixing to a level in range between pH 3 and pH 8, preferably in range from pH 5 to pH 7.5.

In a preferred embodiment buffer is used in the measurement for standardization of the pH. Preferably, the buffer is non-chelating, zwitterionic Good's buffer, such as HEPES or tris-bis propane. The pH of the buffer solution is adjusted to a suitable range, preferably to pH 5-7.5. The pH should not be excessively alkaline in order to prevent possible precipitation of the lanthanide hydroxides.

Unknown hydrolysis degree of the polyelectrolyte or phosphonate in the sample is determined from the measurement by comparing the sample signal to calibration curve. The calibration standard samples have known hydrolysis degree and known concentration. The calibration curve is produced by measurement of known samples having known hydrolysis degrees. The known samples should have same polyelectrolyte or phosphonate concentration as the unknown samples. The hydrolysis degree of the samples (both calibration samples and unknown samples) may vary e.g. in the range of 1-100 mol-%, preferably in the range of 5-60 mol-%.

The unknown charge density of the polyelectrolyte or phosphonate in the sample is determined from the measurement by comparing the sample signal to the calibration curve. The calibration standard samples have known charge density and known concentration. The calibration curve is produced by measurement of known samples having known charge densities. The known samples should have same polyelectrolyte or phosphonate concentration as the unknown samples.

The lanthanide(III) ion is excited at excitation wavelength and measured at emission wavelength and detected by using time-resolved fluorescence (TRF). Any TRF reader can be employed. Excitation and emission wavelengths are selected so that the S/N is the best. Also the delay time can be optimized.

The excitation and emission wavelengths and the delay time are chosen based on the requirements of the lanthanide ion.

In an exemplary embodiment 250-400 nm can be used as excitation wavelength region and 575-625 nm can be used as emission wavelength region for Europium.

In another exemplary embodiment excitation wavelength and emission wavelength and delay time for Europium is 395 nm and 615 nm and 400 μs respectively.

The present invention further relates to use of the method of the present invention for determining hydrolysis degree of polyelectrolyte or phosphonate or charge density of polyelectrolyte or phosphonate in a sample.

The sample can originate from water treatment, paper making processes, pharmaceutical industry, well drilling, mineral processing, enhanced oil recovery, an oilfield or an oil well or from an oil production process.

The present invention further relates a device comprising means for performing the method according to the present invention for determining hydrolysis degree or charge density of polyelectrolyte or phosphonate in a sample.

The examples are not intended to limit the scope of the present invention, but to present embodiments of the present invention.

### EXAMPLES

Polymers (polyacrylamide or polyacrylate) are dissolved into brine. The brines contain alkaline and earth alkaline metals as chlorides or bicarbonates. The TDS of the brines varies between 20 000 and 40 000 ppm. The diluted polymer samples are eluted through GE NAP-10 column to UV cuvette. HEPES buffer solution (adjusted to pH 7.4) and EuCl₃ is added into the cuvette. The concentrations of polymer in the measurement vary between ˜3 and 17 ppm. The concentrations of HEPES and Eu are 5 mM and 10μM in the measurement mixture. The samples are measured with TRF reader. The lag time, excitation and emission wavelengths used were 401 μs, 396 nm and 615 nm, respectively.

Table 1 presents hydrolysis degree measurements of polyacrylamides. TRF signal of polyacrylamides with varying polymer concentration (3.3-16.7 ppm in the measurement mixture) and hydrolysis degree (20-60 mol-%), and 3 parallel measurements are measured for each sample and concentration. In the last column, the TRF signal of the sample is compared to that of 40 mol-% hydrolyzed polymer sample.

TABLE 1

| Polyacrylamide | | | | |
|---|---|---|---|---|
| concentration | | | | Recovery-% |
| in the measurement | Hydrolysis | Photon | | (Signal/40% |
| mixture (ppm) | degree | count | AVERAGE | polymer signal) |
| 3.3 | 60 | 11675 | 11345 | 116.4 |
| | | 11447 | | |
| | | 10914 | | |
| | 40 | 9417 | 9750 | 100 |
| | | 9765 | | |
| | | 10069 | | |
| | 25 | 3337 | 3209 | 32.9 |
| | | 3161 | | |
| | | 3129 | | |
| | 21.6 | 2007 | 1994 | 20.4 |
| | | 1926 | | |
| | | 2048 | | |
| 16.7 | 60 | 12835 | 11806 | 40.3 |
| | | 10018 | | |
| | | 12564 | | |
| | 40 | 30179 | 29279 | 100 |
| | | 27280 | | |
| | | 30377 | | |
| | 25 | 9141 | 9138 | 31.2 |
| | | 9452 | | |
| | | 8822 | | |
| | 21.6 | 3447 | 3685 | 12.6 |
| | | 3552 | | |
| | | 4055 | | |
| 13.3 | 40 | 26730 | 26186 | 100 |
| | | 27798 | | |
| | | 24031 | | |
| | 25 | 8682 | 8576 | 32.7 |
| | | 8044 | | |
| | | 9002 | | |
| | 21.6 | 3222 | 3433 | 13.1 |
| | | 3544 | | |
| | | 3533 | | |

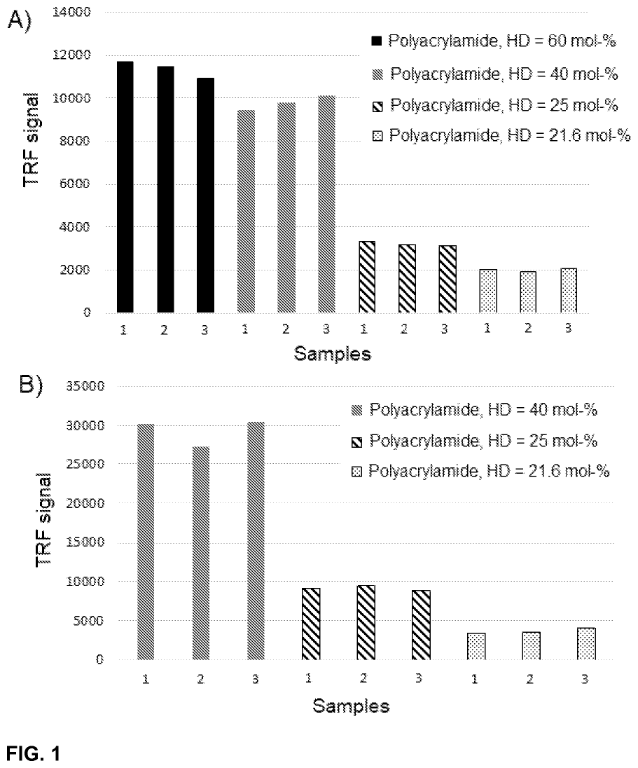

FIG. 1 presents TRF response of polyacrylamides with different hydrolysis degree. Sample concentrations in the measurement mixture are A) 3.3 ppm: B) 16.7 ppm.

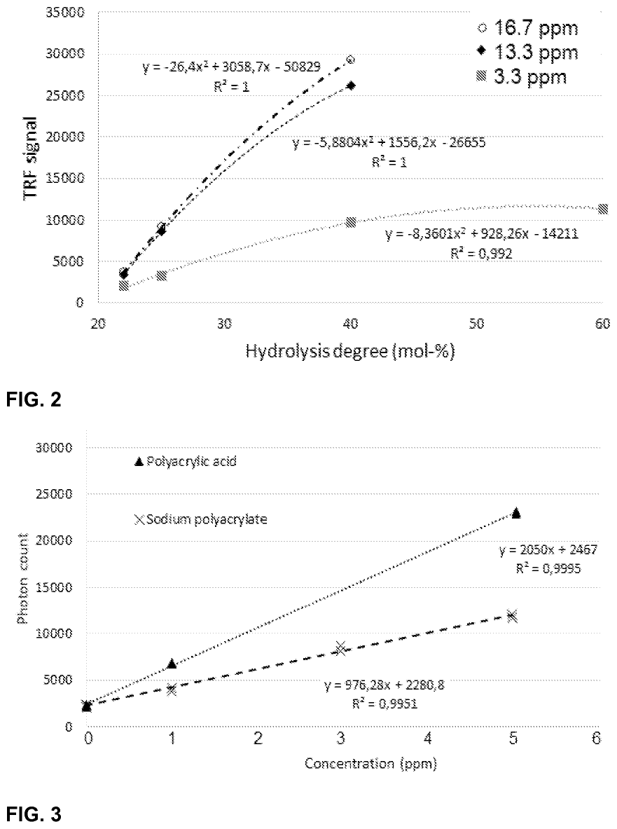

FIG. 2 presents TRF signals (calibration curves) as a function of hydrolysis degree of polyacrylamide. Average results from the three parallel measurements are presented in the FIG. 2. It can be seen that TRF signal depends on the hydrolysis degree and polymer concentrations (the concentrations presented are concentrations in the measurement mixture).

FIG. 3 presents TRF signals of polyacrylic acid (pH 6.66) and sodium polyacrylate (pH 3.90) (calibration curves). The TRF signal decreases when the fraction of polyacrylate fraction increases. The signal change is utilized for charge measurement of polyacrylic acids/polyacrylates. It can be seen that the TRF signal depends on the concentration polyacrylic acid and sodium polyacrylate fractions. The concentrations presented in the FIG. 3 are concentrations in the measurement mixture.

1. A method for determining hydrolysis degree or charge density of polyelectrolyte or phosphonate in a sample comprising polyelectrolyte or phosphonate, the method comprising:
optionally diluting and/or purifying the sample;
admixing the sample with a reagent comprising a lanthanide(lll) ion;
allowing the polyelectrolyte or phosphonate in the sample to interact with the reagent comprising the lanthanide(lll) ion;
exciting the sample at an excitation wavelength and detecting a sample signal deriving from the lanthanide(lll) ion at a signal wavelength by using time-resolved fluorescence measurement; and
determining the hydrolysis degree or the charge density of the polyelectrolyte or phosphonate in the sample by using the detected sample signal.

2. The method according to claim 1, wherein the polyelectrolyte contains one or more groups selected from ester, ether, carboxylic acid/carboxylate, amide, phosphonate, amine groups or any combination thereof.

3. The method according to claim 1, wherein the method is for determining of hydrolysis degree of polyelectrolytes or phosphonate.

4. The method according to claim 1, wherein the method is for determining of charge density of polyelectrolytes or phosphonate.

5. The method according to claim 1, wherein the reagent comprising a lanthanide(lll) ion is a lanthanide(lll) salt.

6. The method according to claim 1, wherein the lanthanide(lll) ion is selected from europium, terbium, samarium or dysprosium ions, preferably europium or terbium ions.

7. The method according to claim 5, wherein the lanthanide(lll) salt is selected from halogenides and oxyanions, preferably from hydrated halogenides or nitrates, more preferably chloride.

8. The method according to claim 1, wherein concentration of the lanthanide(lll) ion in the measurement mixture is in the range of 0.01-100 μM, preferably 0.1-20 μM, and more preferably 1-20 μM.

9. The method according to claim 1, wherein concentration of the polyelectrolyte or phosphonate in the measurement mixture is in the range of 0.01-100 ppm, preferably 0.1-50 ppm, and more preferably 0.5-20 ppm.

10. The method according to claim 1, wherein a signal modifier is added to the sample before the excitation of the sample, preferably the signal modifier comprises a metal ion selected from the group consisting of copper, nickel, chromium, iron, gold, silver, cobalt, and any of their mixtures.

11. The method according to claim 1, wherein the sample is purified by using a purification method selected from centrifugation, size exclusion chromatography, cleaning with solid-phase extraction (SPE) cartridges, dialysis techniques, extraction methods for removing hydrocarbons, filtration, microfiltration, ultrafiltration, nanofiltration, membrane centrifugation and any combinations thereof.

12. The method according to claim 1, wherein a pH value of the sample is adjusted to a level in range between pH 3 and pH 8, preferably in range from pH 5 to pH 7.5.

13. Use of the method according to claim 1 for determining hydrolysis degree of polyelectrolyte or phosphonate or charge density of polyelectrolyte or phosphonate in a sample.

14. The use according to claim 13, wherein the sample originates from water treatment, paper making processes, pharmaceutical industry, well drilling, mineral processing, enhanced oil recovery, an oilfield or an oil well or from an oil production process.

15. A device comprising means for performing the method according to claim 1 for determining hydrolysis degree or charge density of polyelectrolyte in a sample.
